(19) United States
(12) Patent Application Publication
(10) Pub. No.: US 2010/0137923 A1
(43) Pub. Date: Jun. 3, 2010

(54) MINIMALLY INVASIVE ORTHOPAEDIC DELIVERY DEVICES AND TOOLS

(75) Inventors: Skott E. Greenhalgh, Wyndmoor, PA (US); John-Paul Romano, Chalfont, PA (US); Michael P. Igoe, Perkasie, PA (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/088,893

(22) PCT Filed: Nov. 9, 2006
(86) PCT No.: PCT/US06/43790

§ 371 Date: Feb. 3, 2010

Related U.S. Application Data
(60) Provisional application No. 60/735,554, filed on Nov. 10, 2005.

(51) Int. Cl. A61B 17/56; A61M 29/00

(52) U.S. Cl. 606/86 R; 606/192

(57) ABSTRACT

A tool for working in a cavity to which access is limited is disclosed. The tool has an elongated sheath with a bore and an open end that is positionable within the cavity. A filamentary element is slidably positioned within the bore of the sheath. A portion of the filamentary element is extendible outwardly from the sheath through the open end and into the cavity. The filamentary element may be biased into a curved or helical shape, and may be a loop or may have an awl, a cutting blade, a scoop, hook, balloon or other piece attached to its end.

### FIELD OF THE INVENTION

This invention relates to surgical tools that provide access to cavities for manipulation of items within the cavities.

### BACKGROUND OF THE INVENTION

During surgical procedures, it is often necessary to work within cavities in bone or other tissue for the installation or manipulation of surgical implants or tools to effect a desired result. It is advantageous to provide access to such cavities with as little trauma to the patient as possible as, for example, is accomplished by means of laparoscopic surgical techniques. However, such techniques limit access to the cavities and therefore require specialized tools that may pass through small incisions in the soft tissue surrounding the cavity and permit work to be accomplished within the cavity.

An example of such a procedure is shown in FIG. 1 which illustrates repair of a fractured femur 10 at the head 12 where it engages the hip bone (not shown). In the procedure, a small incision is made in the leg that provides access to the femur, and a cavity 14 is excavated in the bone adjacent to head 12. An insertion tool 16 is then used to insert a fitting 18 into the cavity. Fitting 18 provides a clear channel through which a bone screw (not shown) may be inserted to engage the head 12 and secure it to the femur 10.

The cavity 14 is larger than necessary to insert the fitting 18 to allow bone cement to be pumped into it. When it cures, the bone cement provides a foundation for the fitting and the bone screw, strengthening the repair. The bone cement is added preferably within a flow control element such as bag 20 that is attached to the fitting, the bag expanding to fill cavity 14 as the bone cement is pumped into it. Flow control elements are not limited to bags and may also include vessels of various types. The bags and vessels may be formed from interlaced filamentary members or thin films, slit films, spun bonded membranes, as well as injection molded chambers. Other types of flow control devices include splines, fins, petals, spars, fingers and the like. The various flow elements may be made of polyester, PEEK, steel, titanium, nitinol, PCL, PGA, nylon, PMMA, acrylics, ceramics thin metal films, bone cement, wax, biological tissues, cadaver tissue (like skin), collagen and elastin to list some examples.

Unfortunately, the bone cement, being viscous, does not cause the bag or other flow control device to reliably deploy within the cavity 14. The bag 20 must be folded around the fitting 18 to pass through the incision and the opening in the bone, and often becomes tangled with itself, the fitting, and parts of the bone, preventing its deployment when the bone cement is injected, even under pressure. It should be noted that media, such as particulate matter, bone chips, BMPs, growth hormones and other compounds, may also be injected in addition to bone cement.

It would be advantageous to have a tool that provides access to the cavity from outside the body that can be used to manipulate the bag 20 and deploy it into the cavity so that the bone cement can be injected so as; to completely fill the cavity and provide a foundation for the repair without voids or discontinuities.

### SUMMARY OF THE INVENTION

The invention concerns a tool for working in a cavity to which access is limited. One embodiment of the tool comprises an elongated sheath having a bore therethrough and an open end positionable within the cavity. A filamentary element is slidably positioned within the sheath. A portion of the filamentary element is extendible outwardly from the sheath through the open end and into the cavity. Preferably, the elongated sheath has an angled tip portion positioned at the open end. The filamentary element may comprise a wire loop that is biased into a predetermined shape. Alternately the filamentary element may comprise a wire biased into a predetermined shape, for example, a helical shape. The wire may have a blunt tip positioned at one end extendible from the sheath or a tool head such as an awl, a cutting blade, a scoop or a hook.

In a further embodiment, the tool includes an inflatable balloon attached to the open end of the sheath, the balloon being in fluid communication with the bore, the bore providing a conduit for conveying pressurized fluid to inflate the balloon.

Additionally, an elongated fin may be attached to the wire proximate to the end extendible from the sheath. The fin engages the sheath for orienting the wire in a predetermined angular orientation relatively to the sheath.

In an alternate embodiment, the tool comprises an elongated sheath having a bore therethrough and an open end positionable within the cavity. An elongated flexible tube is slidably positioned within the sheath. The tube has an end portion extendible outwardly from the sheath through the open end. The tube is biased into a predetermined curved shape which it assumes when extended from the sheath. A filamentary element is slidably positioned within the tube. A portion of the filamentary element is extendible outwardly from the tube and into the cavity.

In another embodiment, the tool comprises an elongated sheath having a bore therethrough and an open end positionable within the cavity. An elongated tube is slidably positioned within the sheath. One end of the tube is positionable proximate to the open end of the sheath, the tube having a bore therethrough for conducting a pressurized fluid therethrough. A balloon is attached to the one end of the tube, the balloon being in fluid communication with the bore and inflatable when the pressurized fluid is conducted through the tube into the balloon. A portion of the balloon extends from the open end of the sheath. The balloon extends outwardly from the sheath upon inflation thereby drawing the tube toward the open end of the sheath. The tool may further include a wick positioned within the balloon. One end of the wick is attached to the tube, the other end of the wick is attached to the balloon, the wick conducting the pressurizing fluid through the balloon.

### BRIEF DESCRIPTION OF THE DRAWINGS

FIG. 1 is a partial sectional view of a fractured femur being repaired by injecting bone cement into a fitting and bag within a cavity within the bone;

FIGS. 2 and 2A show a partial sectional view of embodiments of a tool providing accessibility to the bone cavity;

FIG. 3 shows a partial sectional view of bone cement being injected into a bag deployed within a bone cavity;

FIG. 4 is a cross-sectional view taken at line 4-4 of FIG. 1;

FIGS. 5-7 are longitudinal sectional views of an embodiment of a tool having an inflatable balloon;

FIGS. 8-10 are sectional views of another tool embodiment having an inflatable balloon;

FIGS. 11 and 12 are cross sectional views taken along lines 11-11 and 12-12 in FIGS. 8 and 8A respectively;

FIGS. 13-15 are sectional views of another embodiment of a tool providing accessibility to a cavity;

FIGS. 16-18 are sectional views of another embodiment of a tool providing accessibility to a cavity;

FIGS. 22-26 are side views of various further embodiments of tools providing accessibility to a cavity;

FIGS. 27-30 are partial sectional views illustrating steps in the use of a tool for attaching a bag within a cavity of a bone;

FIGS. 31 and 32 are partial sectional views illustrating a tool according to the invention being used in the treatment of a vertebral fracture;

FIGS. 33 and 34 are side views illustrating a tool according to the invention being used to replace a herniated disc between vertebrae;

FIGS. 35-37 are partial sectional views illustrating a tool according to the invention being used to treat a fracture of a long bone; and

FIGS. 38-40 are partial sectional views illustrating a tool according to the invention being used to treat a bone fracture.

### DETAILED DESCRIPTION OF PREFERRED EMBODIMENTS

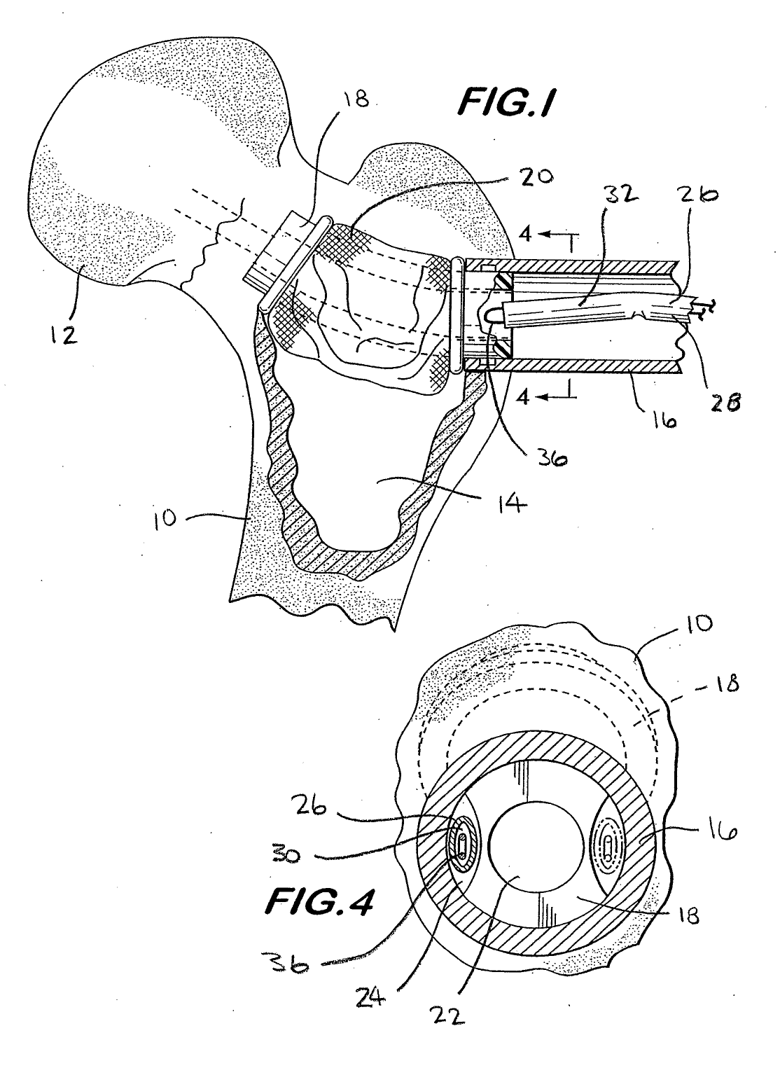

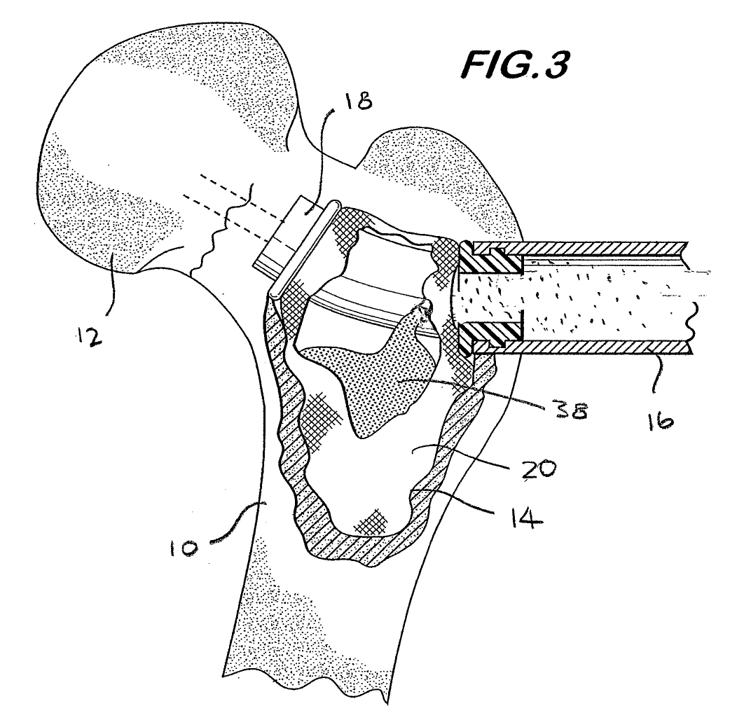

As shown by way of example in FIG. 1, a repair to a fractured femur 10 is effected by excavating a cavity 14 in the femur near the fractured head 12. Access to the bone is provided by a small incision that permits an insertion tool 16 to pass through muscle tissue to the bone cavity 14. A fitting 18 is releasably attached to the end of insertion tool 16 and is inserted into the cavity 14 where it acts as an insert or anchor to attach a bone screw (not shown) between the head 12 and the remainder of the femur 10. A bag 20 is folded around the fitting 18 allowing both items to pass through the incision and into the cavity 14. As shown in FIG. 4, fitting 18 has a bore 22 that receives the bone screw. A pair of access ports 24 are positioned on opposite sides of the bore 22. The access ports are in fluid communication with the interior of bag 20 and allow bone cement to be pumped into the bag to provide the foundation for the repair, securing the fitting 18 to the bone as shown in FIG. 3.

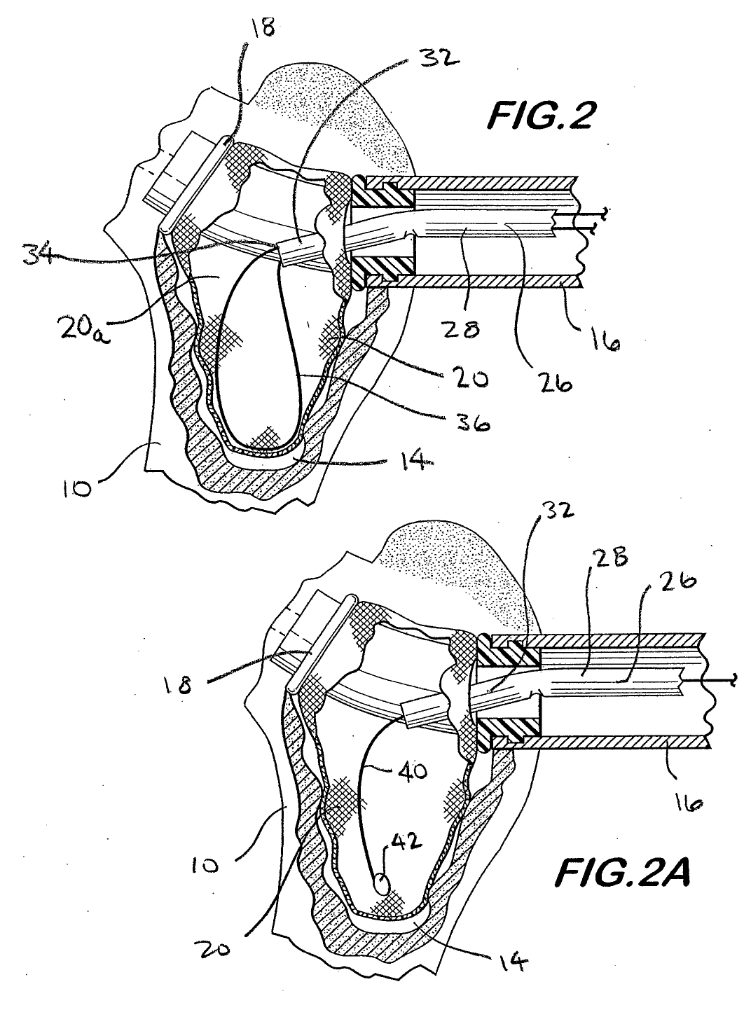

However, pumping the bone cement into bag 20 cannot reliably ensure that the bag will expand to fill cavity 14 without voids due to the viscosity of the cement and the behavior of the bag, which can become tangled and hung up on itself, as well as the fitting and the bone. As shown in FIG. 2, the difficulty of deploying the bag is overcome using the cavity access tool 26. Tool 26 comprises an elongated sheath 28 sized to fit within insertion tool 16 and be manipulated from outside of the patient. Preferably, sheath 28 is formed of metal such as titanium, aluminum or stainless steel compatible with living tissue and has an oval or elliptical cross section defining a bore 30 as shown in FIG. 4. Polymer materials such as engineering plastics are also feasible for forming the sheath. The shaped cross section allows the sheath to pass through access port 24 and into the interior space 20a defined by bag 20. Sheath 28 may also have an angled tip 32 allowing its open end 34 to be conveniently aimed downwardly into cavity 14. In the embodiment shown in FIG. 2, a wire loop 36 is slidably positioned within bore 30 of sheath 28. Once the tip 32 is properly positioned within bag 20, the wire loop 36 is extended from the sheath. The loop is formed of metal such as titanium, stainless steel, nitinol, elgiloy or other bio-compatible materials. The loop 36 has sufficient stiffness to apply force to the bag as it extends outwardly from sheath 28 and move the bag from its folded configuration (FIG. 1) into a deployed configuration as shown in FIG. 2. Use of shape memory metals such as nitinol and elgiloy is advantageous as the loop may be configured to assume a particular shape or radius of curvature appropriate to the size and shape of the bag 20 and cavity 14. The loop may be a monofilament, a multifilament, or of braided construction, and may range from about 0.015 inches to about 0.030 inches in diameter. Once the bag is fully deployed the loop 36 may be retracted and the sheath withdrawn from insertion tool 16. As shown in FIG. 3, bone cement 38 may then be pumped through the insertion tool into the bag 20 to fill the cavity 14 and secure fitting 18 to the bone in repair of the fractured head 12.

FIG. 2A shows an alternate embodiment of the cavity access tool 26 that uses a single wire 40 with a blunt “atraumatic” tip 42. Wire 40 is slidable within sheath 28 and may be biased into a predetermined curved shape that directs it downwardly into the cavity 14, the wire assuming its curved shape as it is extended from the sheath. Again, the wire exerts a force on the bag, deploying it into cavity 14 from its folded configuration. The blunt tip 42 may be a plastic or metal bulb attached to the end of the wire and ensures that the bag is not punctured and the bone is not chipped or otherwise damaged.

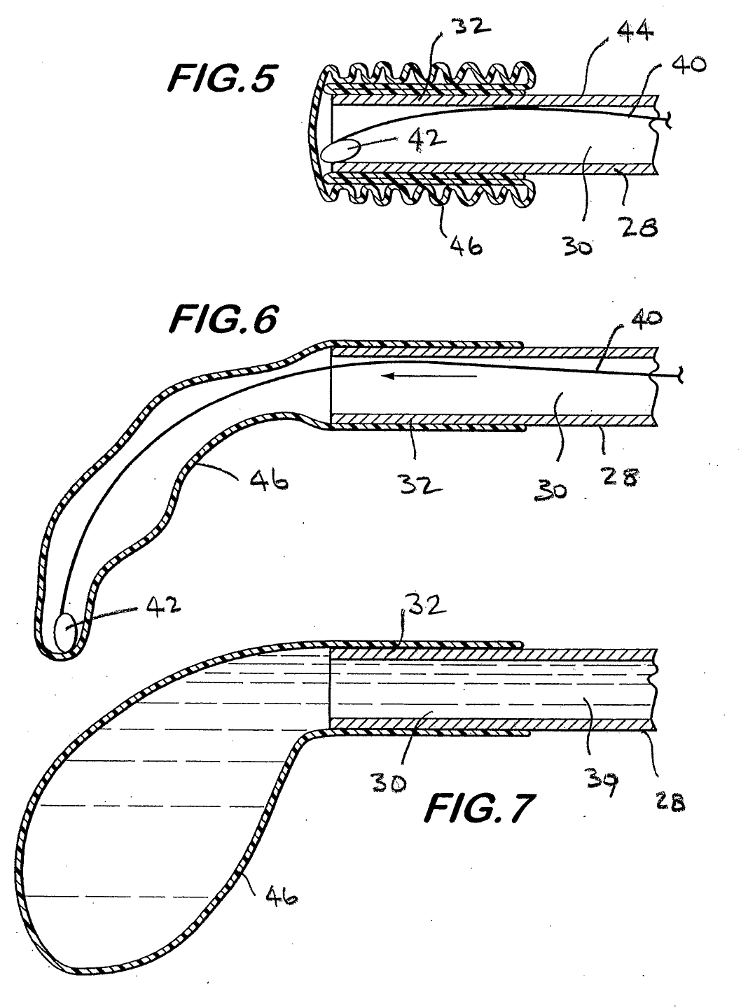

FIG. 5 illustrates another embodiment 44 of the cavity access tool. A balloon 46 is attached to sheath 28, which again has a bore 30 that receives a slidable wire 40 with blunt tip 42. The balloon 46 may be compliant and assume the shape of the bag and cavity as it is inflated, semi-compliant or non compliant, meaning that it will assume a predetermined shape and not expand into a different shape regardless of how much pressure is applied to inflate it. The balloon may be formed from materials such as latex, silicone, polyisoprene and urethane, for example, and may be reinforced with fabric scrim.

In operation, the balloon is in fluid communication with bore 30 and folded around the tip 32 of sheath 28 as shown in

FIG. 5. Once positioned as desired, for example within a cavity such as a bone being repaired, the wire 40 is extended from sheath 28 as shown in FIG. 6, pushing the balloon from its folded configuration into an extended configuration. Wire 40 may then be withdrawn and fluid 39 pumped through bore 30 to inflate the balloon as shown in FIG. 7. The balloon may, thus, be used to unfold a bag within a cavity as described above. Unlike the wire 40, the balloon provides a three-dimensional shape for manipulating the bag within the cavity to ensure its full deployment in all directions.

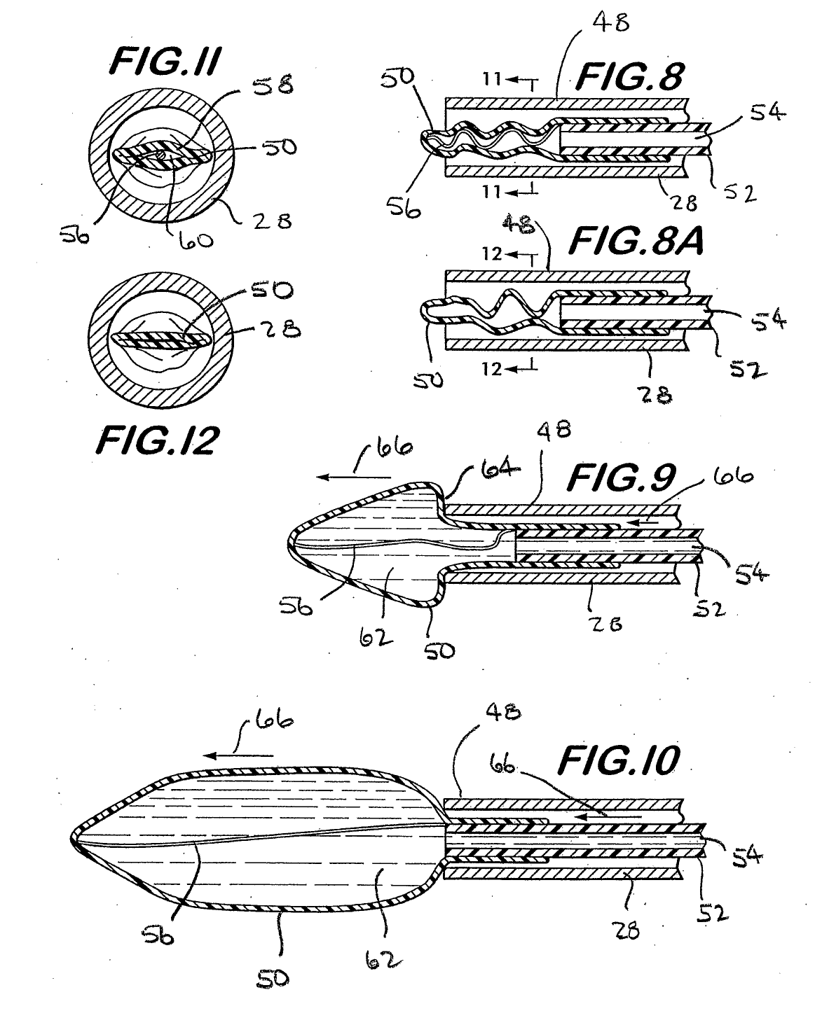

FIG. 8 illustrates a cavity access tool 48 that uses a non-compliant balloon 50 to exert an axial force within an otherwise inaccessible cavity. Balloon 50 is preferably formed from materials such as PET, PEBAX and nylon to achieve the non-compliant properties desired. The balloon 50 is attached to a tube 52 that is received within sheath 28. Tube 52 has a bore 54 in fluid communication with balloon 50, allowing fluid to be pumped into the balloon to inflate it. As shown in FIGS. 8 and 11, a wick 56 may be positioned within the balloon. One end of the wick is attached to the tube 52, the other end being attached to the balloon. As shown in FIG. 11, wick 56 keeps the balloon sidewall 58 separate when it is folded and provides pathways 60 through the length of the balloon that form on either side of the wick. Using capillary action to guide the fluid, the wick prevents the balloon from resisting inflation by kinking, the pathways 60 allowing inflation fluid to reach every part of the balloon. Alternately, as shown in FIGS. 8A and 12, the wick may be absent from the balloon if not needed.

In operation, as shown in FIG. 8, the tube 52 is within sheath 28 with a portion of balloon 50 extending outwardly from the open end 34. The sheath is positioned within a cavity and fluid 62 is then pumped through tube 52 inflating balloon 50, as shown in FIG. 9. The balloon inflates, and preferably the sheath 28 is held fixed. A shoulder 64 forms between the balloon sidewall 58 and the sheath 28 as the balloon inflates. This causes the balloon to deploy outwardly from the sheath, exerting an axial force as indicated by arrows 66 in FIGS. 9 and 10, drawing the tube into the sheath.

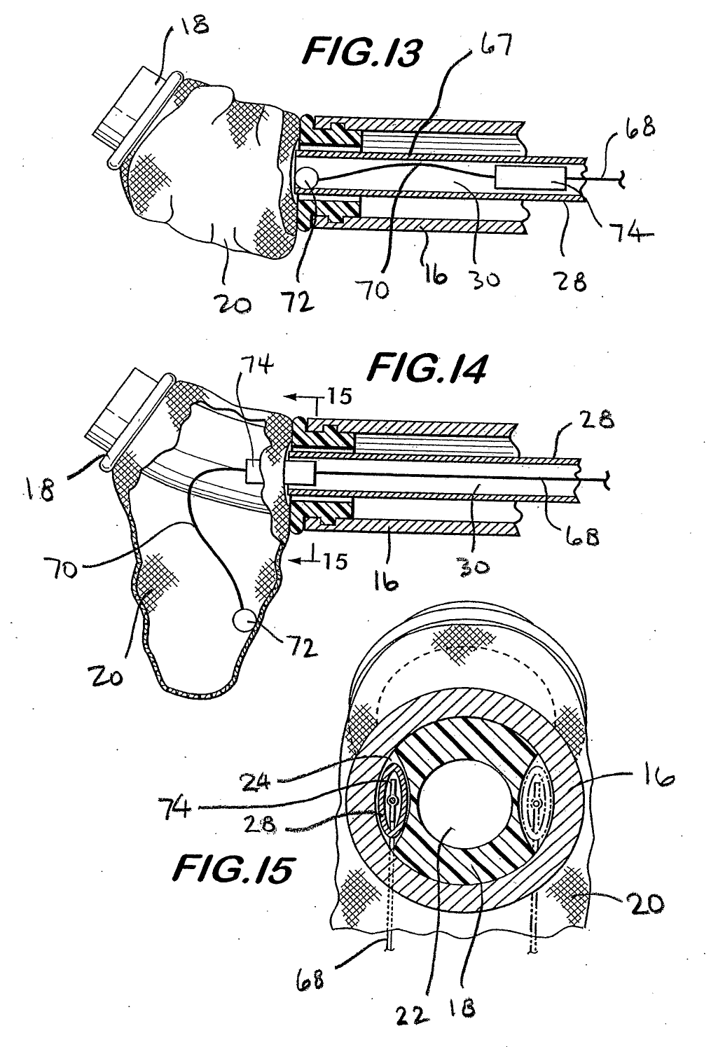

FIG. 13 shows another embodiment of a cavity access tool 67 again comprising an elongated sheath 28 insertable through insertion tool 16 proximate to a bag 20. A wire 68 is slidable along the bore 30 of sheath 28. Wire 68 is formed of a material such as spring steel and has an end portion 70 that is biased into a predetermined shape that is assumed once the end is free of the constraints of the sheath as illustrated in FIG. 14. Again a blunt end 72 is attached to the wire so that it does not puncture the bag when deployed. Preferably, the biased shape of the wire end 70 is designed to accommodate the size and shape of the bag 20 and the cavity in which it is deployed. The spring force due to the biasing of the wire facilitates deployment of the bag. To ensure that the shaped end 70 is properly oriented so as to exert forces that deploy the bag 30 into the cavity, an orienting fin 74 is attached to the wire adjacent to the shaped end 70. As shown in FIG. 15, the fin 74 engages the sheath 28 or the access ports 24 and prevents the wire 68 from rotating, thus orienting the shaped end 70 so that it forces the bag 20 into the deployed configuration, in this example downwardly, extending the bag away from its fitting 18.

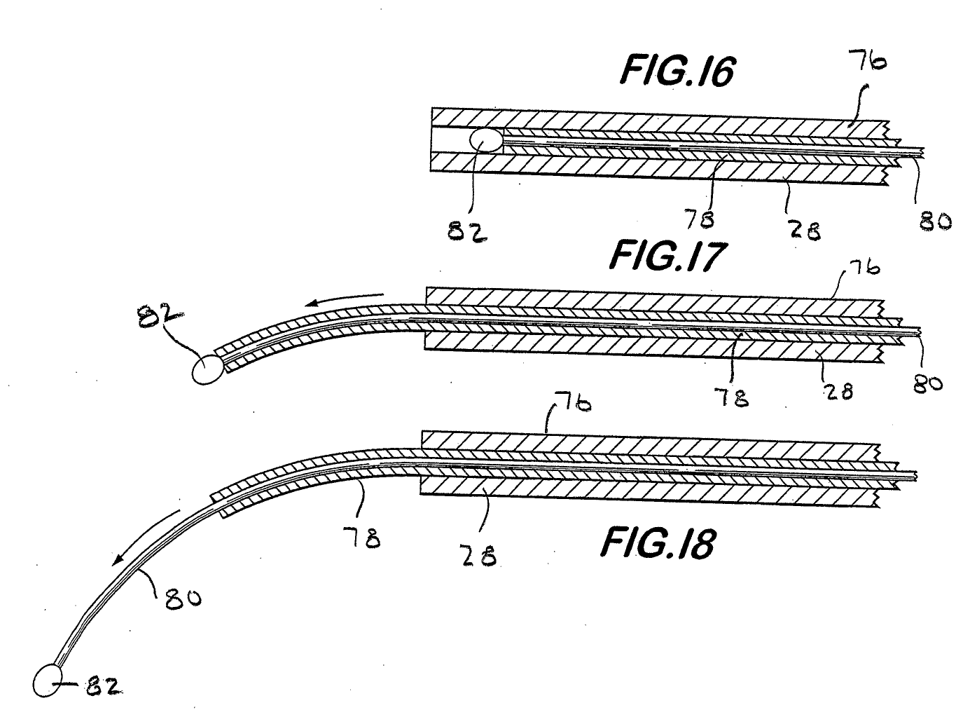

It is sometimes desired to provide a cavity access tool having increased stiffness and greater tactile feed back over the embodiments already described. Such a tool embodiment 76 is shown in FIG. 16 and comprises a sheath 28 that surrounds a tube 78 slidable within the sheath. A wire 80 is slidable within the tube 78. To increase the stiffness of the tool, the wire is preferably a metal formed of a material such as stainless steel, titanium, nitinol or elgiloy having a high elastic modulus and a high yield stress. The diameter of wire 80 is preferably between about 0.030 and 0.070 inches. Tube 78 is also preferably a metal of similar material. Both the tube and the wire are biased into a curved shape which they assume when extended out from sheath 28 as shown in FIGS. 17 and 18. Note that the wire and tube may be deployed independently of one another in a telescoping manner. The curvature of the tube 78 and the wire 80 allows the wire tip to be steered in a desired direction that varies from the axial direction of sheath 28 to deploy a bag. A blunt end 82 allows the wire to be used without fear of puncture.

Although wire 80 is shown as a monofilament, it could also be woven or braided, as shown in FIGS. 19-21. Furthermore, wire 80 may also be biased into a three dimensional shape, such as a helix 84. Once free of the constraints of tube 78, the wire 80 expands in three dimensions in to the spiral shape to facilitate expansion of the bag into a three-dimensional volume.

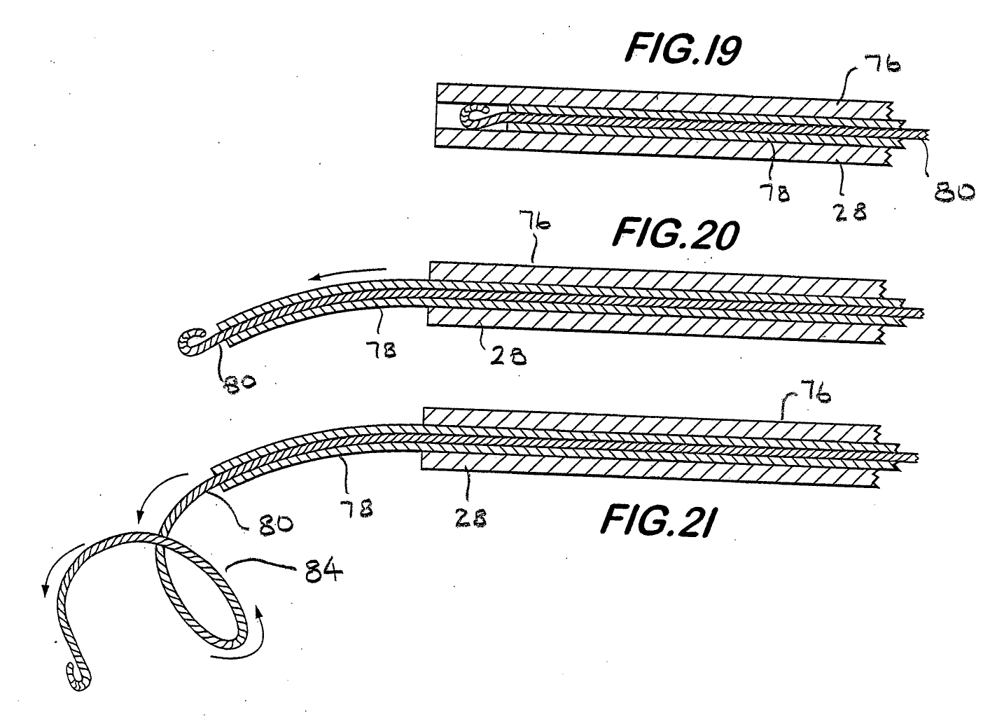

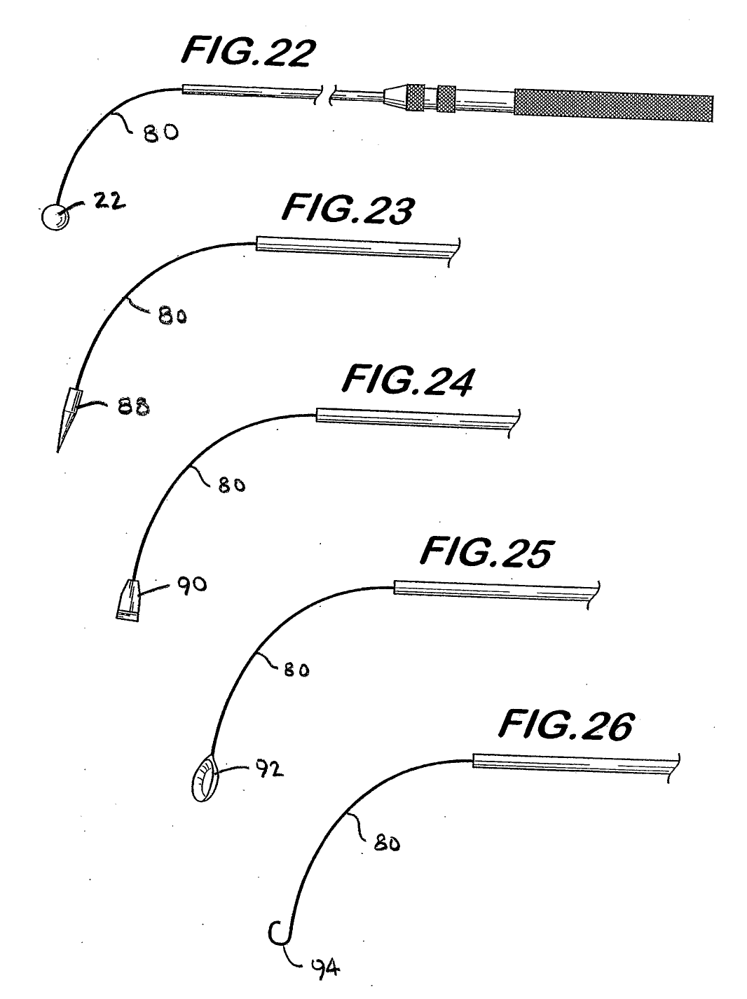

Although blunt “atraumatic” tips 86 as shown in FIG. 22 have been described in the various embodiments discussed thus far, other tips for performing other functions are also feasible. As shown in FIG. 23, a pointed penetrating tip 88 may be attached to a wire 80 allowing the tool to be used as a awl to form a hole or opening. FIG. 24 shows a blade 90 mounted on wire 80 allowing the tool to perform a scraping, chiseling or cutting function. FIG. 25 shows a scoop 92 mounted on wire 80 for removing matter, and FIG. 26 shows a hook 94 for snagging items.

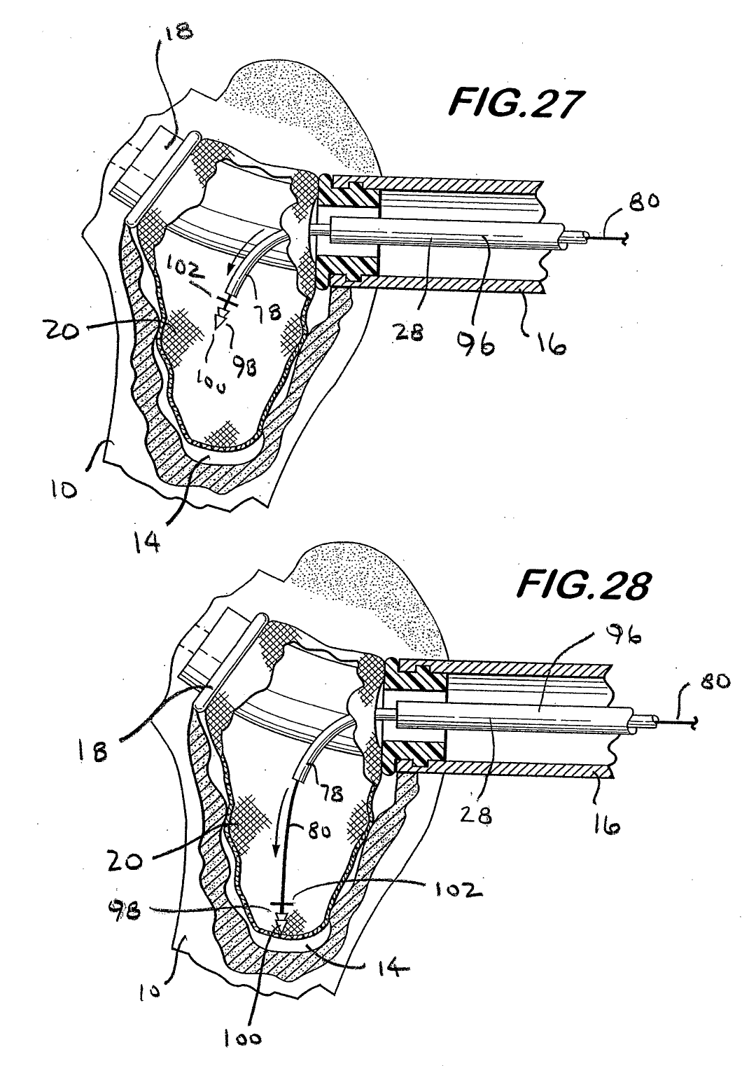

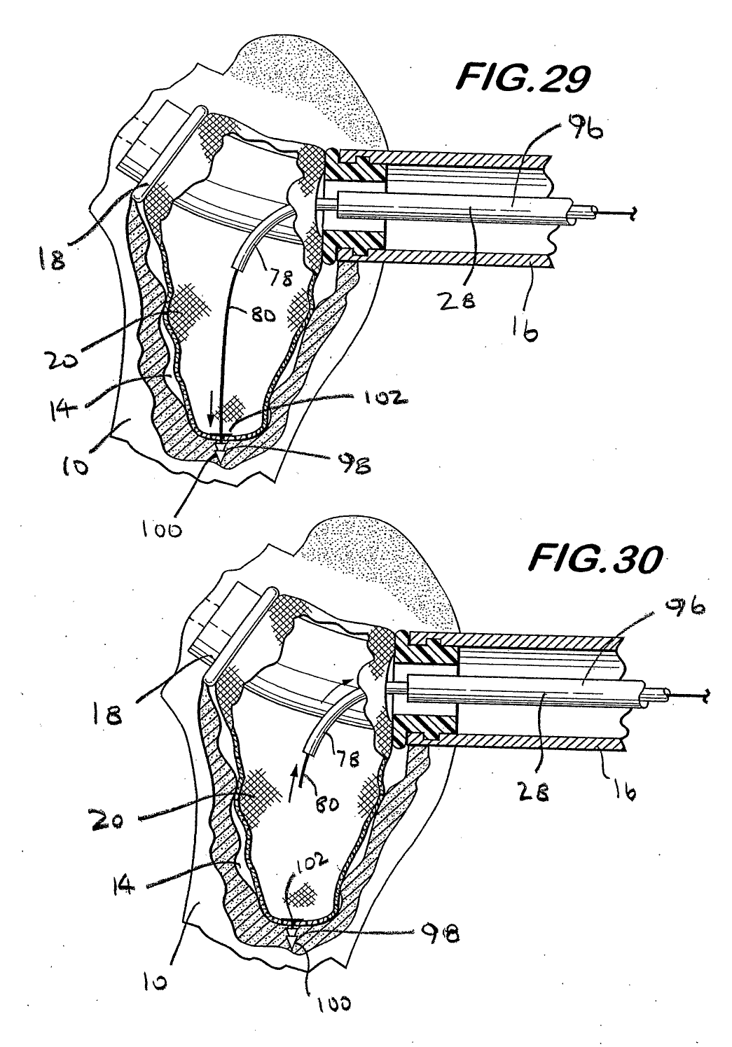

The cavity access tool according to the invention may also be used to deliver items to a cavity. As shown in FIG. 27, a fitting 18 with a bag 20 is deployed within a bone cavity 14 to effect a repair of a fracture. As described previously, a tool is used to help deploy the bag into the cavity. However, it may be desirable to attach the bag to the bone before the cement is pumped in. To this end, a tool 96 having a barbed fastener 98 releasably mounted on the end of a wire 80 constrained within a tube 78 which itself is slidable within a sheath 28 is inserted through insertion tool 16. The tube 78 is extended out from sheath 28 and assumes its biased curved shape, pointing the fastener 98 towards the bottom of bag 20. As shown in FIG. 28, the wire 80 is then extended from the tube 78, and the barbed tip 100 of the fastener is driven through bag 20 and into the bone material as shown in FIG. 29. Opposite to the barbed tip is a retaining shoulder 102 that engages the bag 20 to retain it to the tip. Once the barbed tip is driven home it is released from wire 80, for example by using a notch in wire 80 that forms a weakened region that can break away when tension is applied. The wire 80 and tube 78 may then be withdrawn into the sheath 28 and the sheath removed to allow bone cement to be pumped into the bag 20 through insertion tool 16.

The various tool embodiments illustrated and described herein are not limited in use to those examples provided above, but are useful in any situation where minimally invasive techniques are required and access to the space required to effect treatment is limited.

A further example of an application for the tools according to the invention is illustrated in FIGS. 31 and 32. FIG. 31 depicts treatment of a fractured vertebral body 104 using a tool 26. Similar to the examples describe above, tool 26 comprises a sheath 28 in which is positioned a slidable element 106, preferably in the form of a flexible wire 108 having an atraumatic blunt tip 110 used to manipulate a bag 112 positioned at the end of the sheath 28 within the fracture of the vertebral body 104. Once the bag is properly positioned, soft filler or bone cement may be injected into the fracture to restore the vertebra to its proper shape as illustrated in FIG. 32. Upon completion of the treatment, the bag 112 is detached from the sheath 28 and the sheath and slidable elements 106 are withdrawn with minimal effect on surrounding tissue.

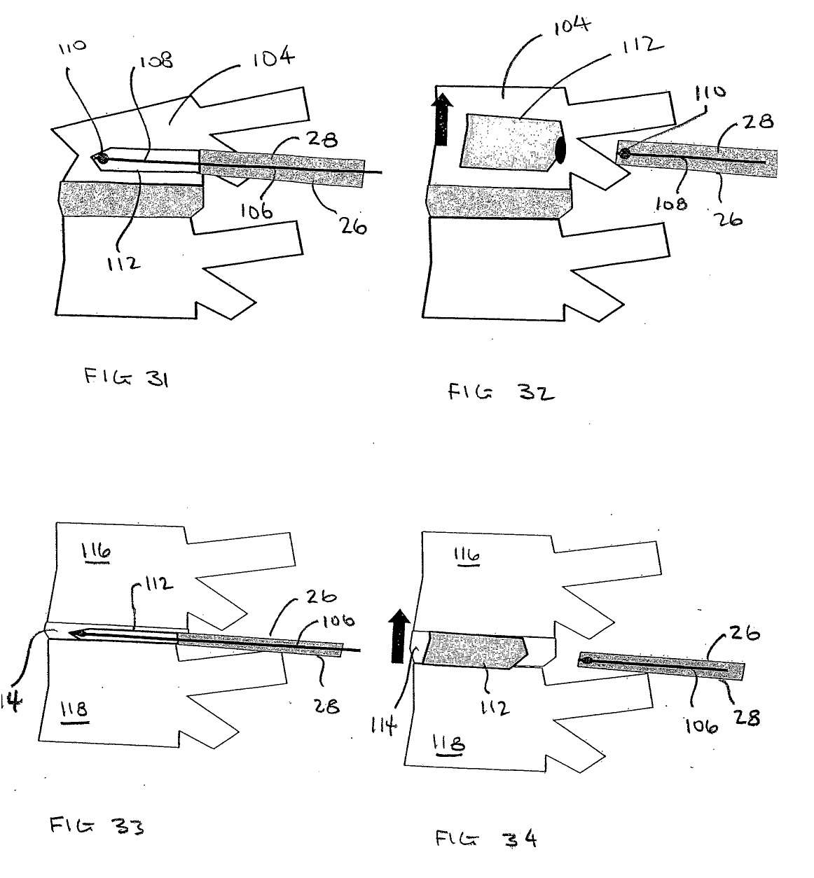

Tools according to the invention are not limited to repairs of fractures, but may also be used to fuse vertebrae where a disc is ruptured or herniated as shown in FIGS. 33 and 34. FIG. 33 shows a tool 26 comprising sheath 28 being positioned adjacent to an intravertebral space 114 whereupon a bag 112 is deployed between the vertebrae 116 and 118 using element 106 slidable within sheath 28. Once deployed, as shown in FIG. 34, bag 112 is filled with bone cement which bonds with the adjacent vertebrae 116 and 118 to fuse the joint.

Repairs or other treatment of spinal disorders using tools according to the invention as described above may be effected over the entire spinal column, from the lumbar to the cervical regions.

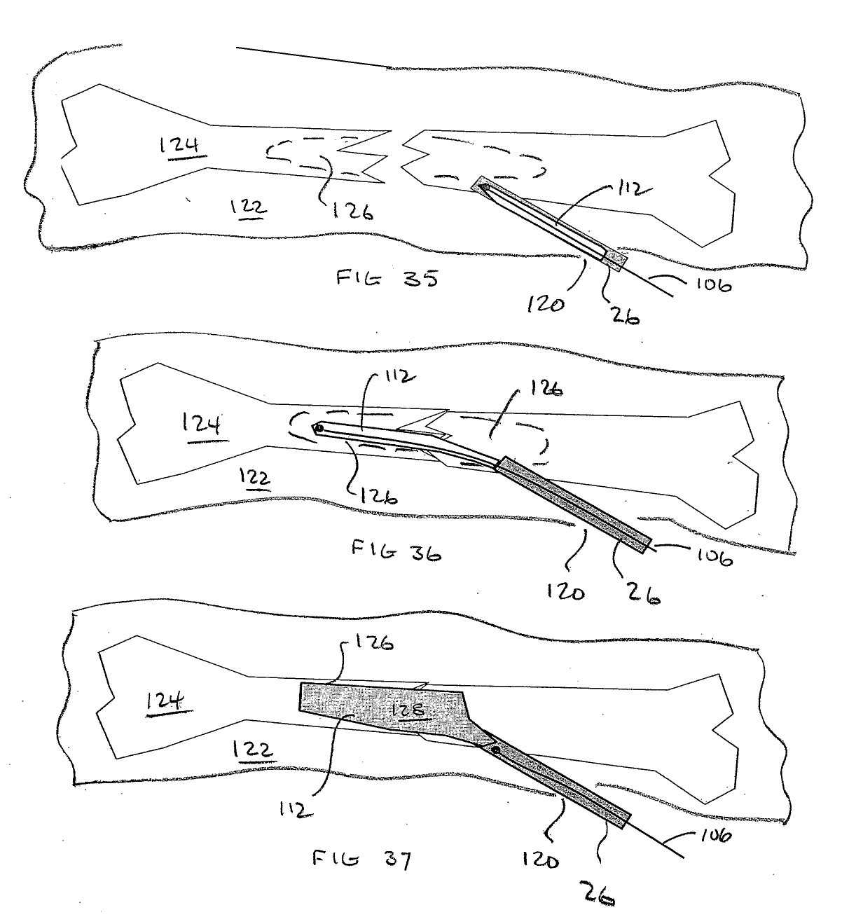

FIGS. 35 through 37 illustrate treatment of a long bone fracture using a tool 26 according to the invention. As shown in FIG. 35, the tool 26 is inserted through a small incision 120 with minimal trauma through living tissue 122 surrounding the fractured bone 124 and positioned adjacent to the fracture. A slidable element 106 positioned within the sheath 28 comprising the tool is extended to manipulate the bag 112 (see FIG. 36) and position it within the marrow cavity 126 of the bone. Once positioned, bag 112 is inflated with bone cement 128 which forms a bridge that holds the bone pieces in alignment with one another allowing them to heal. The tool is removed through the incision, again with minimal trauma as shown in FIG. 37.

FIGS. 38 through 40 illustrate another embodiment 130 of a tool according to the invention. Again, the application is treatment of a fractured bone 124. As shown in FIG. 38, tool 130 is inserted through an incision 132 in living tissue 134 surrounding the fracture. Tool 130 includes a sheath 136 in which multiple slidable elements 138 are positioned. In this example there are two slidable elements 138a and 138b, but there could also be more than two. Again, as described above, the slidable elements may comprise flexible wire loops or wires having blunt, atraumatic ends 140 as shown, or barbed ends, or an implement, such as a scraper, hook, or chisel.

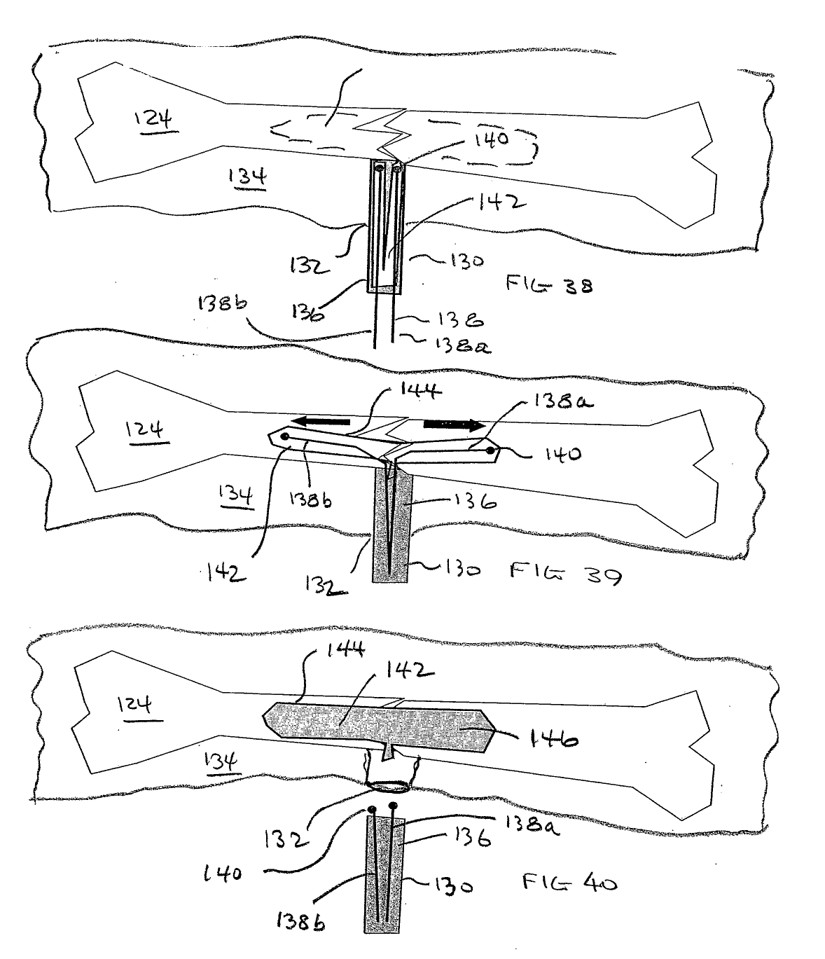

FIG. 39 illustrates the advantage of multiple slidable elements 138, which allow a bag 142 positioned at the end of sheath 136 to be manipulated in opposite directions into the marrow cavity 144 of the bone 124. Once the bag is properly deployed, bone cement 146 or other bio-compatible filler material may be pumped into the bag to form an aligning bridge to fix the bone fragments in place and allow them to heal. As shown in FIG. 40, the tool 130 may be withdrawn upon completion of the treatment with little or no trauma to the surrounding tissue.

Although illustrated used to position fabric bags within a cavity having limited access, the tool embodiments according to the invention may also be used to manipulate and position non-fabric items such as balloons, membranes, thin films, porous films and the like.

Cavity access tools as described herein allow work to be performed within a cavity that has limited accessibility. Such tools provide an advantage when used, for example, in surgery, in that trauma to a patient is minimized because the tools according to the invention may work within a cavity through small incisions.

What is claimed is:

1. A tool for working in a cavity having limited access, said tool comprising:
an elongated sheath having a bore therethrough and an open end positionable within said cavity; and
a filamentary element slidably positioned within said sheath, a portion of said filamentary element being extendible outwardly from said sheath through said open end and into said cavity.

2. A tool according to claim 1, wherein said elongated sheath has an angled tip portion positioned at said open end.

3. A tool according to claim 1, wherein said filamentary element comprises a wire loop.

4. A tool according to claim 3, wherein said wire loop is biased into a predetermined shape.

5. A tool according to claim 1, wherein said filamentary element comprises a wire.

6. A tool according to claim 5, wherein said wire is biased into a predetermined shape.

7. A tool according to claim 6, wherein said wire is biased into a helical shape.

8. A tool according to claim 5, wherein said wire has a blunt tip positioned at one end extendible from said sheath.

9. A tool according to claim 1, further comprising an inflatable balloon attached to said open end of said sheath, said balloon being in fluid communication with said bore.

10. A tool according to claim 5, wherein said wire has a tool attached at one end extendible from said sheath.

11. A tool according to claim 10, wherein said tool is selected from the group consisting of an awl, a cutting blade, a scoop and a hook.

12. A tool according to claim 5, further comprising an elongated fin attached to said wire proximate to said end extendible from said sheath, said fin engaging said sheath for orienting said wire in a predetermined angular orientation relatively to said sheath.

13. A tool for working in a cavity having limited access, said tool comprising:
an elongated sheath having a bore therethrough and an open end positionable within said cavity;
an elongated flexible tube slidably positioned within said sheath, said tube having an end portion extendible outwardly from said sheath through said open end, said tube being biased into a predetermined curved shape, said tube assuming said curved shape when extended from said sheath; and
a filamentary element slidably positioned within said tube, a portion of said filamentary element being extendible outwardly from said tube and into said cavity.

14. A tool for working in a cavity having limited access, said tool comprising:
an elongated sheath having a bore therethrough and an open end positionable within said cavity;
an elongated tube slidably positioned within said sheath, one end of said tube being positionable proximate to said open end of said sheath, said tube having a bore therethrough for conducting a pressurized fluid; and
a balloon attached to said one end of said tube, said balloon being in fluid communication with said bore and inflatable when said pressurized fluid is conducted through said tube into said balloon, a portion of said balloon extending from said open end of said sheath, said balloon extending outwardly from said sheath upon inflation thereby drawing said tube toward said open end of said sheath.

15. A tool according to claim 14, further comprising a wick positioned within said balloon, one end of said wick being attached to said tube, the other end of said wick being attached to said balloon, said wick conducting said pressurizing fluid through said balloon.
